(19) United States
(12) Patent Application Publication
(10) Pub. No.: US 2010/0266669 A1
(43) Pub. Date: Oct. 21, 2010

(54) MULTI-ZONE FILMS

(75) Inventors: Stephan Meyer, Geneve (CH); Greg Slominski, Valparaiso, IN (US); Christopher Edward Fankhauser, Eagle, NE (US); Nicole Ouis, Renens (CH)

Correspondence Address: Novartis Consumer Health, Inc., OTC Patent Group, 200 Kimball Drive, Parsippany, NJ, 07054-0622, US

(73) Assignee: Novartis AG, Basel (CH)

(21) Appl. No.: 12/746,614

(22) PCT Filed: Dec. 9, 2008
(86) PCT No.: PCT/EP2008/067074

§ 371 Date: Jun. 7, 2010

Related U.S. Application Data
(60) Provisional application No. 61/007,192, filed on Dec. 11, 2007.

(51) Int. Cl. A61K 31/465; A61K 9/70; B05D 1/34

(52) U.S. Cl. 424/444; 514/343; 427/2.14

(57) ABSTRACT

The invention relates to single-layer oral disintegrating films having at least two distinct zones, which comprise nicotine and allow for effective buccal (=oral mucosal) absorption thereof.

The invention relates to a new oral dosage form for the administration of nicotine in smoking cessation therapy. More specifically, it concerns oral disintegrating films which comprise nicotine and which completely disintegrate in the mouth of a patient typically within 1 to 10 minutes, preferably within 2 to 6 minutes. Further, said oral disintegrating films are composed of at least two different film compositions, which at least two different film compositions form at least two distinct zones of the films that exist together to comprise a single unit.

Typically, a single-layer film is aimed at. But one can also imagine bi- or multilayer films which include at least one layer formed according to the present invention.

Well known oral dosage forms comprising nicotine and being used in smoking cessation therapy are e.g. chewing gums and lozenges. Typically, it is intended therein that nicotine is absorbed through the buccal mucosa. However, chewing gums and lozenges have turned out to be dosage forms which usually are releasing nicotine rather slowly and thus are not ideal to promote rapid transmucosal absorption.

Within this document, the term “buccal mucosa” is used synonymously to “oral mucosa” and is intended to cover the mucosa of the entire oral cavity.

It was found that nicotine dosage forms with an early high absorption peak do mimic the smoking of a cigarette best, do not present insurmountable toxicity issues and thus are often preferable in smoking cessation therapy, especially to reduce tobacco craving. Therefore, one of the goals of the present invention was to provide an oral nicotine dosage form which provides for a high rate of buccal (=oral mucosal) absorption of nicotine and thus for a rapid, high plasma peak for nicotine.

Oral disintegrating films comprising pharmaceutically active substances represent a relatively new oral dosage form and have only recently reached the first markets. Many of them, when put into the mouth, dissolve immediately, so that the active is readily swallowed and “normal” gastro-intestinal absorption takes place.

The present inventors, when experimenting with such films comprising nicotine, found that nicotine in free base form was too reactive and volatile to remain within the film during the manufacturing process and upon storage. Thus, it turned out that nicotine in salt form or in a “bound” form had to be used, preferably as a pharmaceutically acceptable nicotine salt. Bound nicotine means e.g. pharmaceutically acceptable nicotine complexes and resins, e.g. nicotine polacrilex.

Upon testing films with nicotine salts or bound nicotine, however, it turned out that no sufficient transmucosal, i.e. buccal, absorption was obtained. This presumably can be explained by the fact that oral pharmaceutical dosage forms containing nicotine salts typically require the presence of an alkaline substance (often also referred to as “pH regulator” or “buffer”) in order to optimize its buccal absorption. It has been assessed that the buccal pH should rise to 8 or above to optimize the transformation of a nicotine salt (which shows little or no transmucosal absorption) into the well buccally absorbed nicotine free base.

Apart from the pH effect, the inventors found that a quick disintegrating film (e.g. one disintegrating in less than 30 sec) seemed not to be well suited to ensure transmucosal absorption of nicotine. Rather, nicotine was largely swallowed with the saliva after disintegration of the film.

Therefore, it is an object of the present invention to provide a slow disintegrating film which has an increased contact time with the buccal mucosa, which releases the nicotine gradually and thus optimizes the buccal absorption of nicotine.

Thus, the oral disintegrating nicotine films according to the present invention typically do disintegrate within 1 to 10 min, preferably 2 to 6 min, after being put into the mouth of a patient.

To reach the goal of increased contact times with the buccal mucosa, it was found that the films typically has to be mucoadhesive (also named “bioadhesive”), i.e. they have to adhere well to the oral mucosa and so prevent the nicotine film composition from being immediately swallowed.

In a preferred embodiment of the invention, the nicotine film is orally taken up by putting it on the tongue, where it typically does not adhere very well. In contact with saliva, it will form a bioadhesive gel, or a wet polymer layer respectively, within seconds, and is typically lifted by the tongue to adhere to the mucosa of the upper part of the oral cavity (hard palate). In another embodiment of the invention, the nicotine film may be placed at the inside of one cheek, e.g. by attaching it with the finger tip. Another possibility would be to place the nicotine film under the tongue (sublingual administration). In principle, the film can be adhered to any of the oral tissues once it has been wetted with saliva and formed a bioadhesive gel, or a wet polymer layer respectively.

Moreover, as mentioned above, there was a need to include an alkaline substance into the film to increase the pH in the oral cavity throughout the disintegration/administration period of the oral film product.

Attempts to combine nicotine salts and alkaline substances in the same phase were not successful, however, mainly due to stability reasons. It was found that when an otherwise stable nicotine salt was used in a single-phase film system that the nicotine salt rather quickly transforms into volatile and unstable nicotine free base.

Therefore, the nicotine active and the alkaline substance need to be physically separated within the oral disintegrating nicotine films of the present invention, so that they will mix only when, after intake, the film is disintegrating and gradually releasing both ingredients into the oral cavity. It is an object of the present invention to physically separate both components in a single layer film product. This has been accomplished by providing a single layer film which has distinct zones, some of which include the nicotine active and some other of which include the alkaline substance. Processes to manufacture said single-layer, bi- or multi-zonal films on an industrial scale are described below.

Edible films having distinct regions, wherein at least one region has a composition that is different from at least one other region are known from US 2004/0120991. But the focus there is on immediately disintegrating edible films only. In contrast thereto, the present invention provides a bi- or multi-zone pharmaceutical film which slowly disintegrates within 1 to 10 minutes and which is composed of typically two different specific compositions that confer optimal properties on the product for efficient and fast buccal absorption of nicotine.

“Efficient and fast buccal absorption of nicotine” typically means reaching a tₘₐₓ, value (=“time to peak plasma concentration”) in the blood plasma of a patient of 30 min or less, or 15 min or less, preferably of from 4 to 30 min, or of from 4 and 15 min, especially of from 10 to 30 min, more especially of from 15 to 30 min, and in particular of from 15 to 25 min.

Unlike the compositions described in patent US 2004/0120991, the present invention describes a pharmaceutical film that transforms itself, within a few seconds when in contact with the saliva, into a bioadhesive gel, or a wet polymer layer respectively, that adheres to the buccal mucosa (e.g. on the cheek or more preferably on the hard palate). Said bioadhesive gel, or wet polymer layer respectively, contains both zones and disintegrates slowly (within 1 to 10 min) thereby gradually releasing both the nicotine salt and the alkaline substance. By doing so, it enables the nicotine salt to be transformed in situ (i.e. within the oral cavity) into nicotine free base as a result of the increased pH due to the presence of the dissolved alkaline substance.

Therefore, the invention relates to a single-layer, oral disintegrating film having at least two distinct zones,

wherein at least one zone (a) consists of a first composition that comprises a nicotine active, and
wherein at least one zone (b) consists of a second composition that comprises an alkaline substance,
and which oral disintegrating film optionally includes one or more further distinct zones selected from the group of zones (a′) and (b′), which zones (a′) and (b′) are characterized by having essentially the same compositions as the corresponding zones (a) and (b), respectively.

Preferably, said films adhere to the buccal mucosa when wetted by the saliva.

Pharmaceutically acceptable nicotine salts are e.g. nicotine bitartrate such as nicotine bitartrate dihydrate, nicotine hydrochloride, nicotine dihydrochloride or nicotine sulfate.

As alkaline substance there comes into consideration any pharmaceutically acceptable excipient that is able to rise the buccal pH, e.g. sodium carbonate, potassium carbonate, sodium bicarbonate, potassium bicarbonate, sodium hydroxide, potassium hydroxide, or any mixture thereof; and preferably sodium carbonate, potassium carbonate, sodium hydroxide, potassium hydroxide, or any mixture thereof.

The amounts of nicotine active and of the alkaline substance (present in one film) can be adjusted via the compositions used for the nicotine active zones (a), (a′) and alkaline substance zones (b), (b′) as well as the size of the final film cut. With these options, it is possible to obtain bi- or multi-zone nicotine films with a release profile as desired, typically a profile ensuring efficient and fast buccal absorption of nicotine. The actual amounts being present in a bi- or multi-zone nicotine film may vary a lot depending on, in particular, the kind of nicotine active and alkaline substance used, and the kind of film forming polymers and other excipients present.

In one preferred embodiment of the invention, a bi- or multi-zone nicotine film has a width of 12 to 30 millimeters and a length of 20 to 50 millimeters, and comprises an equivalent of 1 to 3, in particular 1 to 2, mg of nicotine base as well as from 8 to 25, in particular 9 to 20, mg of sodium carbonate.

The zones (a) and (a′) typically include at least one water soluble film forming polymer, e.g. cellulose, cellulose ether derivatives, synthetically or naturally occurring gums, polyalkylene oxides, polyalkylene glycols; acrylic acid polymers, acrylic acid copolymers, methacrylic acid polymers, methacrylic acid copolymers, polyacrylamides, pullulan, polyvinyl pyrrolidone, polyvinyl alcohol, polyvinyl alcohol copolymers e.g. polyethylene glycol-polyvinyl alcohol copolymers, modified starch, amylose, high amylose starch, hydroxypropylated high amylose starch, dextrin, pectin, chitin, chitosan, levan, elsinan, collagen, gelatin, zein, gluten, soy protein isolate, whey protein isolate or casein.

It should be noted, that within the definition of water soluble film forming polymers above there are listed some groups of polymers which can be either water soluble or non-water soluble depending e.g. on their detailed chemical structure or on the pH value that is applied. It is considered to be within the knowledge of the man skilled in the art to properly select a corresponding water soluble—or non-water soluble—film forming polymer from the list above.

Non-water soluble film forming polymers are e.g. ethyl cellulose and certain methacrylic acid copolymers, especially copolymers derived from esters—in particular alkyl, aminoalkyl and ammonioalkyl esters—of methacrylic and acrylic acid, e.g. from the Eudragit® series [supplied e.g. by Evonik Roehm GmbH (Darmstadt, Germany)], especially Eudragit® L, S, FS, E, RL or RS polymers (with acidic or alkaline groups) or, in particular, Eudragit® NE polymers (with neutral groups, e.g. methyl or n-butyl ester groups). For selecting non-water soluble film forming polymers, special emphasis is on ethyl cellulose and ethyl acrylate methyl methacrylate copolymers, such as Eudragit® NE 30 D or Eudragit® NE 40 D.

In one embodiment of the invention, the zones (a) and (a′) include at least two different water soluble film forming polymers selected from the list above.

Suitable water soluble cellulose ether derivatives include alkyl celluloses e.g. methyl cellulose, substituted alkyl celluloses e.g. hydroxyethyl cellulose, hydroxypropyl cellulose, hydroxypropyl methylcellulose or carboxymethyl cellulose, and salts of substituted alkyl celluloses e.g. sodium carboxymethyl cellulose, and mixtures thereof. Preferred is hydroxypropyl methylcellulose (=HPMC).

Suitable synthetically or naturally occurring gums include xanthan gum, tragacanth gum, guar gum, acacia gum, arabic gum, alginic acid, salts of alginic acid e.g. sodium alginate, dammar gum, gellan gum, gucomannan gum, carrageenan gum, ghatti gum, karaya gum, locust bean gum, tara gum and mixtures thereof. Preferred is xanthan gum.

A polyalkylene oxide is e.g. polyethylene oxide, polypropylene oxide, polybutylene oxide or a copolymer thereof, and in particular polyethylene oxide.

The total amount of water soluble film forming polymer(s) is e.g. from 30-95%—preferably from 40-90% and in particular from 45-90%—of the dry film composition of zones (a) and (a′).

In one embodiment of the invention, the zones (a) and (a′) include at least one water soluble film forming polymer selected from the group consisting of cellulose ether derivatives and at least one water soluble film forming polymer selected from the group consisting of synthetically or naturally occurring gums and polyalkylene oxides. In said case, the weight ratio of cellulose ether derivatives to synthetically or naturally occurring gums/polyethylene oxide in zones (a) and (a′) is typically from 15:1 to 2:1, and preferably from 6:1 to 2:1.

In a preferred embodiment of the invention, the zones (a) and (a′) include a combination of at least one water soluble film forming polymer with at least one non-water soluble film forming polymer, e.g. ethyl cellulose or a copolymer derived from alkyl, aminoalkyl or ammonioalkyl esters of methacrylic and acrylic acid.

Without wishing to be bound by theory, it is assumed that the presence of non-water soluble film forming polymers helps to delay the disintegration of the oral disintegrating films in the mouth in the desired manner, i.e. typically within 1 to 10 minutes. The same function can be fulfilled by certain water soluble film forming polymers, especially swellable ones like e.g. synthetically or naturally occurring gums and polyalkylene oxides as already described above.

In an even more preferred embodiment of the invention, the zones (a) and (a′) include a combination of polyvinyl pyrrolidone with a non-water soluble cellulose ether derivative, e.g. ethyl cellulose. In particular preferred is the combination of polyvinyl pyrrolidone, a non-water soluble cellulose ether derivative, e.g. ethyl cellulose, and hydroxypropylmethyl cellulose—optionally together with a polyalkylene oxide.

In another preferred embodiment of the invention, the zones (a) and (a′) include a combination of polyvinyl pyrrolidone with an ethyl acrylate methyl methacrylate copolymer e.g. Eudragit® NE 30 D. Eudragit® NE 30 D is a 30% aqueous suspension of poly(ethyl acrylate-methyl methacrylate), supplied e.g. by Evonik Roehm GmbH (Darmstadt, Germany). In particular preferred is the combination of polyvinyl pyrrolidone, an ethyl acrylate methyl methacrylate copolymer and a non-water soluble cellulose ether derivative, e.g. ethyl cellulose.

Generally it is preferred to configure the compositions of zones (b), (b′) as similar as possible to those of zones (a), (a′) in order to avoid any rheological problems, especially blurred zones at the interfaces, upon manufacture of the film sheets (see e.g. Example 3). “[A]s similar as possible” in this context means, especially, a similar content of solids, in particular the same mix of film-forming polymers. Further, it means e.g. similar drying kinetics of both compositions, which can be achieved e.g. by appropriate selection of the solvents used.

Thus, the zones (b) and (b′) typically include at least one water soluble film forming polymer selected from the same list of water soluble film forming polymers as mentioned above for zones (a) and (a′).

Suitable non-water soluble film forming polymers include the same materials as mentioned above for zones (a) and (a′).

Suitable cellulose ether derivatives and synthetically or naturally occurring gums include the same materials as mentioned above for zones (a) and (a′).

In one embodiment of the invention, the zones (b) and (b′) include at least one water soluble film forming polymer selected from the group consisting of cellulose, cellulose ether derivatives, polyalkylene glycol copolymers, polyvinyl alcohol and polyvinyl alcohol copolymers, and at least one water soluble film forming polymer selected from the group consisting of synthetically or naturally occurring gums.

For example, the zones (b) and (b′) may include a combination of at least one water soluble film forming polymer selected from the group consisting of cellulose ether derivatives—in particular hydroxypropylmethyl cellulose—and at least one water soluble film forming polymer selected from the group consisting of synthetically or naturally occurring gums.

In said embodiments, the synthetically or naturally occurring gums are preferably present in an amount of from 0.1 to 20%, in particular 0.3 to 5%, by weight of the total composition of zones (b) and (b′).

In another embodiment of the invention, the zones (b) and (b′) further comprise a polymer selected from the group consisting of croscarmellose sodium, corn starch, and any mixtures thereof; in particular croscarmellose sodium. In said case, the zones (a) and (a′) optionally may also further comprise a polymer selected from the group consisting of croscarmellose sodium, corn starch, and any mixtures thereof; in particular croscarmellose sodium.

In a preferred embodiment of the invention, the zones (b) and (b′) include a combination of at least one water soluble film forming polymer with at least one non-water soluble film forming polymer, e.g. ethyl cellulose or a copolymer derived from alkyl, aminoalkyl or ammonioalkyl esters of methacrylic and acrylic acid.

In an even more preferred embodiment of the invention, the zones (b) and (b′) include a combination of polyvinyl pyrrolidone with a non-water soluble cellulose ether derivative, e.g. ethyl cellulose. In particular preferred is the combination of polyvinyl pyrrolidone, a non-water soluble cellulose ether derivative, e.g. ethyl cellulose, and hydroxypropylmethyl cellulose—optionally together with a polyalkylene oxide.

In another preferred embodiment of the invention, the zones (b) and (b′) include a combination of polyvinyl pyrrolidone with an ethyl acrylate methyl methacrylate copolymer e.g. Eudragit® NE 30 D. In particular preferred is the combination of polyvinyl pyrrolidone, an ethyl acrylate methyl methacrylate copolymer and ethyl cellulose.

In another preferred embodiment of the invention, the alkaline substance, e.g. sodium carbonate, is kept in suspension—rather than in solution—in the compositions of zones (b), (b′). The positive effects thereof are (1) that interactions of the alkaline substance with the film-forming polymers present are minimized and (2) that the alkaline substance does not recrystallize upon drying, which might lead to an inhomogeneous alkali content. Keeping the alkaline substance in suspension is achieved by using a solvent composition wherein the alkaline substance is not or only poorly soluble, e.g. mixtures of lower alcohols (e.g. isopropanol or ethanol) and water with a water content of less than 20% by weight, or lower alcohols alone.

If a said mixture of lower alcohols (e.g. isopropanol or ethanol) and water with a water content of less than 20% by weight, or lower alcohols alone are used as solvent for the compositions of zones (b), (b′), the latter preferably comprise polyvinyl pyrrolidone, especially in an amount of 20% by weight or more (e.g. 20-40%) of the total dry mass of the composition.

Other components that may be present in the composition of zones (a), (a′) as well as of zones (b), (b′), are e.g. plasticizers. As plasticizers, there come into consideration, for example, polyalcohols, e.g. glycerol, polyethylene glycol, ethylene glycol or propylene glycol; glycerol monoesters with fatty acids such as n-octanoic acid or oleic acid; sorbitol, polysorbate 80 [=polyoxyethylene (20) sorbitan monooleate], triethyl citrate, acetyl triethyl citrate, tributyl citrate, diethyl phthalate or dibutyl sebacate.

Preferred as plasticizers are glycerol, polyethylene glycol, ethylene glycol, propylene glycol, triethyl citrate, or any mixture thereof; and in particular glycerol.

The plasticizer is typically present in amounts ranging from 0.1 to 15—preferably from 1 to 8 and even more preferably from 1.5 to 7—weight-% of the final edible film (dry mass). In a particular embodiment of the invention, the plasticizer (B) is glycerol and is present in amounts ranging from 1 to 12—preferably 1 to 7, and more preferably 1.5 to 6—weight-% of the film (dry mass).

Moreover, the compositions of zones (a), (a′). as well as of zones (b), (b′), optionally include usual auxiliaries as known in the art, such as, for example, flavors, sweeteners, antioxidants, stabilizers, coloring agents, solubilizing agents and preservatives.

In general, it is preferred that the subdivision of the bi- or multi-zone films of the invention into several zones is not visible to the user. This can be accomplished e.g. by using no coloring agents or by using the same coloring agents for all of the zones of the film.

However, coloring agents can be useful e.g. to add appeal to the product and to aid in the manufacturing of bi- or multi-striped film sheets (see below). Therefore, in another embodiment of the invention, coloring agents are added to either the first or the second composition, or different coloring agents to both, so that a film is obtained wherein the color of the zones (b) and optionally (b′) is different from the color of the zones (a) and optionally (a′), wherein the color of the zones (a) and optionally (a′) is the same, and wherein the color of the zones (b) and optionally (b′) is the same.

Exemplary coloring agents include natural food colors and dyes suitable for food, drug and cosmetic applications, e.g. those colorants known as FD&C (“Food, Drug & Cosmetics”, USA) dyes and lakes. Preferred are water-soluble coloring agents, e.g. FD&C Blue No. 1 (=E133), FD&C Blue No 2, FD&C Green No. 3, Fast green FCF, Chlorophyllis (=E140), Green S (=E142), Quinoline Yellow (=E104), Sunset yellow FCF (=E110). Pigments, e.g. titanium dioxide, come also into consideration as coloring agents.

### Method to Prepare a Striped Film Sheet

A film-forming mixture (intended to form zones a and a′) is prepared by mixing e.g. at least one water soluble polymer and a nicotine salt. Next, e.g. at least one water soluble polymer and an alkaline substance are mixed to form a second homogenous mixture (intended to form zones b and b′). Next, the two film-forming mixtures are cast in stripes, each having a pre-determined width (at least two thereof but theoretically limited only by machine capabilities, i.e. may well be 10 or more, e.g. 16, 32 or 64 stripes), at the same time to a desired thickness (joint coating). These stripes contact each other at the edge, merge together into one or more neat interfaces and form one single, integral film sheet.

Joint coating means to coat by any possible coating technology. For example, by knife-over-roll metering, slot die coating or other, a group of stripes of alternate compositions a and b is effected (in the width).

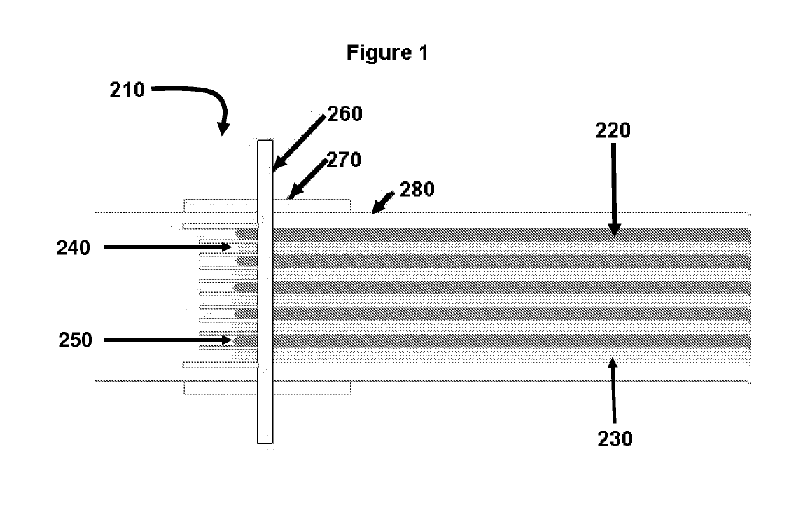

In the case of knife-over-roll technology, the film-forming mixtures of a and b are pumped in equal dimensional compartments placed in front of the knife. The web moving forwards pushes mixtures a and b under the knife, the knife acting as the metering device to determine coating thickness. The cast stripes of product a and b merge in a neat interface before being dried. FIG. 1 shows a top view of a knife-over-roll coating apparatus (210). Coating mix A (220) and coating mix B (230) are pumped into region 250 and 240 respectively while the knife (260) is drawn across the roll (270) toward the direction of the regions 240 and 250. A liner (280) is kept on a plane under the knife (260).

In the case of slot die coating, the coating head is specially designed with one cavity per product. The film-forming mixtures of a and b are each pumped in their proper cavity. The slits for mixtures a and b are alternatively occluded with an appropriate piece having the same width as a stripe. The stripes are cast onto the moving web.

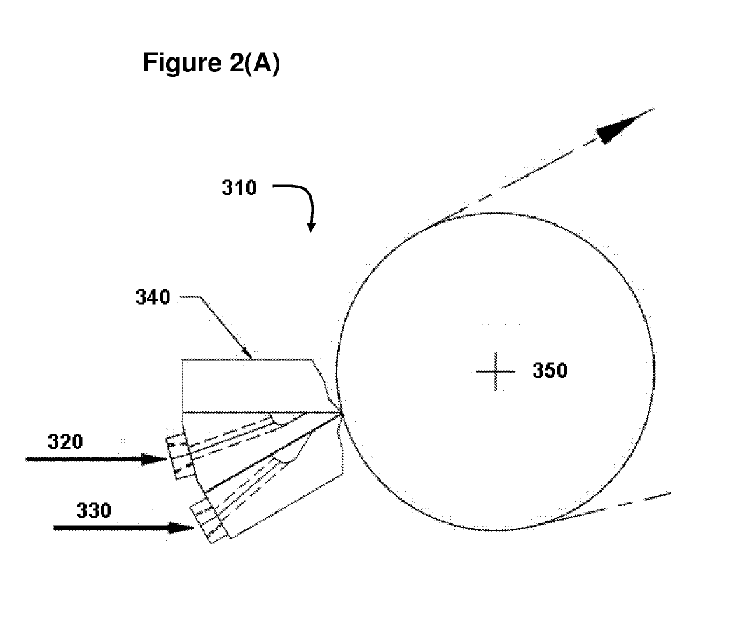

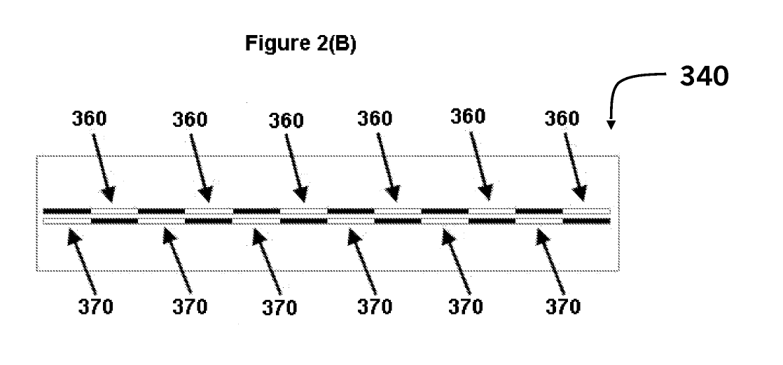

FIG. 2(A) shows a side schematic view of a dual slot die apparatus 310. The apparatus coats a striped film on a rotating coating roll 350. Coating mix A 320 and coating mix B 330 are injected into separate cavities in dual slot die 340.

FIG. 2(B) shows a front view of a dual slot die 340. Coating mix A exits from the open coating mix A slots 360. Coating mix B exits from the open coating mix B slots 370. The remaining coating mix slots blacked out in FIG. 2(B) are closed.

Said single, integral film sheets represent another embodiment of the invention. Thus, the invention also relates to a single, integral film sheet which is longitudinally divided at least once into at least one stripe composed of a first composition that comprises a nicotine active and another at least one stripe composed of a second composition comprising an alkaline substance; wherein the different compositions composing each stripe have merged together.

Typically, casting of the film sheet requires the use of a carrier. The mixtures are cast on a releasable carrier and dried. The carrier material must have adequate properties, which allows the film mixtures to spread evenly across the intended carrier width without soaking and forming a destructive bond between the film and carrier substrates. Examples of suitable carrier materials include PET (polyethylene terephthalate), paper or siliconized versions of any of these. Said carrier materials can optionally be coated with a film release product, e.g. a silicone derivative or a fluoropolymer. Drying of the film sheet may be carried out e.g. at high temperature using a drying oven, drying terminal, vacuum drier, or any other suitable drying equipment which does not adversely affect the ingredients of which the film sheet is composed. The dried film sheet may be coiled up on a bulk master roll.

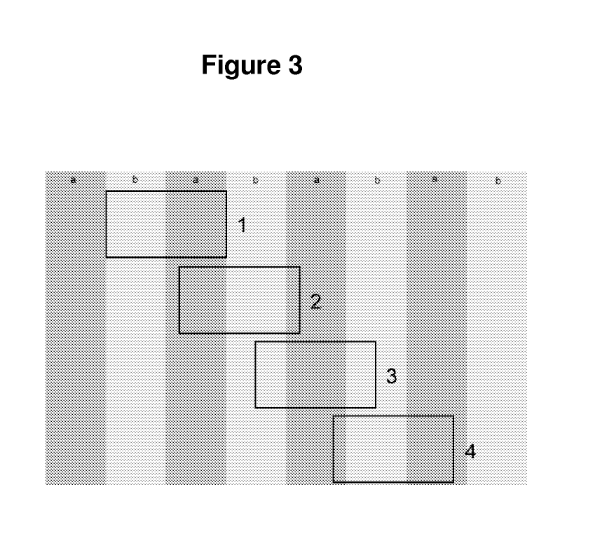

To obtain the bi- or multi-zone films of the invention, the film sheet obtained is cut into pieces, i.e. dosage forms, which may have any form that is suitable for delivery of the present invention, e.g. strips, rectangles, squares or circles. Cutting is performed by die-cutting, slitting-and-die-cutting, laser cutting, or any other technique well known and used in the art. The cutting must be such that bi- or multi-zone films of the invention are obtained, i.e. that each unit dosage form contains the correct amount of nicotine active and alkaline substance. This is accomplished e.g. by cutting out each single dose unit form from at least two adjacent stripes of the film sheet, see e.g. FIG. 3.

In FIG. 3, a film sheet with eight stripes of equal width is shown, with four stripes composed of a and four stripes composed of b. In this example, the correct amounts of nicotine active and alkaline substance for one dosage form are obtained, if a square with a width of two stripes and a defined length is cut (see “1”). In this setting, the correct amounts of nicotine active and alkaline substance are also guaranteed, if the square is not cut exactly over the width of two stripes but cut from three stripes (see “2”, “3” and “4”).

Generally, the bi- or multi-zone films of the invention have a size adapted to the size of the human buccal cavity, e.g. representing a rectangular film of typically 10 to 40, preferably 12 to 30, millimeters in width and typically 15 to 60, preferably 20 to 50, millimeters in length. The bi- or multi-zone films typically have a thickness ranging from 15 to 300 micrometers, and preferably 30 to 150 micrometers.

The invention therefore relates to a method of preparing an oral disintegrating film, the method comprising the steps of:

mixing all components intended to form zones (a) and optionally (a′) to form a first homogenous mixture;
mixing all components intended to form zones (b) and optionally (b′) to form a second homogenous mixture;
feeding said first and second homogenous mixtures separately via at least two different inlets into a coating device capable of spreading each composition into a uniform thin layer while allowing for the separate zones to interface, creating one integral coating with at least two unique stripes; whereby a film is obtained that is longitudinally divided at least once into at least one stripe composed of said first homogenous mixture and another at least one stripe composed of said second homogenous mixture; the different coating mixes composing each zone do merge together during the casting into a neat interface and forming a single, integral film sheet;
drying the cast film; and cutting the produced film into oral disintegrating films such that each oral disintegrating film has at least two distinct zones, at least one of which is composed of said first homogenous mixture and at least one other of which is composed of said second homogenous mixture.

### EXAMPLES

Example 1: Homogeneous mixture of (a), (a′) is prepared (for nicotine active zones)
Example 2: Homogeneous mixture of (b), (b′) is prepared (for alkaline substance zones)
Examples 3, 5, 5a, 7, 8, 9 and 10: Joint coating of both mixtures of Examples 1 and 2 to form film sheet that is longitudinally divided into several stripes (e.g. 8 or 16)
Examples 4, 6 and 6a: Cutting of film sheets so as to obtain bi- or multi-zone single-layer films

In the following Examples 1 and 2, the amounts of all components as indicated are those present in one final film as being cut in Examples 4, 6 and 6a. Likewise, the amounts of solvents are calculated for the manufacture of just one final film. In practice, mixtures are prepared to produce a multitude of final films, thus the amounts given have to be always multiplied with the actual number of films to be prepared.

### Example 1a

### Preparation of a Casting Mixture Containing Nicotine Bitartrate Dihydrate [for Zones (a), (a′)]

451 g of purified water was placed into a stainless steel pot and heated on a hot plate to 80° C. with mixing. To the water solution, 0.03 g of FD&C blue coloring agent, 23.55 g of POLYOX N80 and 47.14 g of METHOCEL E50 and were added and was mixed at a high mixing speed. The stainless steel pot was removed from the hot plate and transferred to a water bath and cooled. Once the mixture had cooled, the stainless steel pot was removed from the water bath and placed into an ice bath and mixed. The stainless steel pot was removed from the ice bath and a menthol solution (10.56 g of menthol in ethanol), 7.54 g of glycerin, 1.95 g of sucralose, 49.53 g of peppermint flavor, and ethyl alcohol was added with mixing. A nicotine solution was prepared by adding 4.01 g of nicotine bitartrate dihydrate to 20 ml of purified water. The nicotine bitartrate dihydrate solution was added to the film casting mixture.

### Composition of Zone in Dry State:

| Ingredient | % in Dry State |
|---|---|
| Polyox N80 (Polyethylene oxide) | 16.32 |
| Methocel E50 (=Hydroxypropylmethyl cellulose, | 32.67 |
| “HPMC”) | |
| Menthol | 7.32 |
| Nicotine bitartrate dihydrate | 2.78 |
| Glycerin | 5.22 |
| Peppermint Flavor | 34.32 |
| Sucralose | 1.35 |
| FD&C Blue | 0.02 |

### Example 1b

### Preparation of a Casting Mixture Containing Nicotine Bitartrate Dihydrate [for Zones (a), (a′)]

| Ingredients | Amount (mg) |
|---|---|
| Methocel E50 (HPMC) | 30.00 |
| Glycerol | 4.00 |
| Xanthan gum | 3.00 |
| Nicotine bitartrate dihydrate (=1 mg nicotine base) | 3.07 |
| Menthol | 1.50 |
| Acesulfame K | 0.50 |
| Mint flavor | 1.00 |
| Total dry mass | 43.07 |
| Purified water | 240.00 |
| Ethanol 96% | 250.00 |
| Total wet mass | 533.07 |

Process: Nicotine bitartrate dihydrate, Acesulfame K and menthol are dissolved in the water-ethanol mix. Glycerol and liquid mint flavor are then added. HPMC and xanthan gum are added slowly under strong stirring. Slow stirring is kept until homogenization of the viscous mixture.

### Example 1c

### Preparation of a Casting Mixture Containing Nicotine Bitartrate Dihydrate (with Croscarmellose Sodium) [for Zones (a), (a′)]

| Ingredients | Amount (mg) |
|---|---|
| Methocel E50 (HPMC) | 30.00 |
| Croscarmellose sodium | 6.00 |
| Glycerol | 3.00 |
| Xanthan gum | 1.00 |
| Nicotine bitartrate dihydrate (=1 mg nicotine base) | 3.07 |
| Menthol | 1.50 |
| Acesulfame K | 0.50 |
| Mint flavor | 1.00 |
| Total dry mass | 46.07 |
| Purified water | 220.00 |
| Ethanol 96% | 140.00 |
| Total wet mass | 406.07 |

Process: Nicotine bitartrate dihydrate, Acesulfame K and menthol are dissolved in the water-ethanol mix. Glycerol and liquid mint flavor is then added. HPMC, croscarmellose sodium and xanthan gum are added slowly under strong stirring. Slow stirring is kept until homogenization of the viscous mixture.

### Example 1d

### Preparation of a Casting Mixture Containing Nicotine Bitartrate Dihydrate [for Zones (a), (a′)]

| Ingredients | Amount (mg) |
|---|---|
| Polyvinyl pyrrolidone (PVP), K-90 | 24.00 |
| Metolose 60 SH 50 (HPMC) | 9.00 |
| Ethyl cellulose | 18.00 |
| Glycerol | 2.10 |
| FD&C Blue No. 1 (colorant) | 0.03 |
| Microcrystalline cellulose | 24.00 |
| Levomenthol | 1.50 |
| Acesulfame K | 0.50 |
| Mint flavor | 0.50 |
| Nicotine bitartrate dihydrate | 3.07 |
| Isopropanol | 150.00 |
| Purified water | 27.00 |
| Total wet mass | 259.70 |
| Total dry mass | 82.70 |

Process: Nicotine bitartrate dihydrate, Acesulfame K, levomenthol and the FD&C Blue No. 1 are dissolved in the water-isopropanol mix. Glycerol, the liquid mint flavor and microcrystalline cellulose are then added. HPMC, ethyl celullose and PVP are added slowly under strong stirring. Slow stirring is kept until homogenization of the viscous mixture.

### Example 1d-2

Example 1d is repeated in an identical manner, except that the amount of nicotine bitartrate dihydrate used is 4.61 mg (=1.5 mg nicotine base equivalent) instead.

### Example 1d-3

Example 1d is repeated in an identical manner, except that the amount of nicotine bitartrate dihydrate used is 5.53 mg (=1.8 mg nicotine base equivalent) instead.

### Example 1d-4

Example 1d is repeated in an identical manner, except that the amount of nicotine bitartrate dihydrate used is 6.14 mg (=2.0 mg nicotine base equivalent) instead.

### Example 1e

### Preparation of a Casting Mixture Containing Nicotine Bitartrate Dihydrate [for Zones (a), (a′)]

| Ingredients | Amount (mg) |
|---|---|
| PVP | 24.00 |
| Metolose 60 SH 50 (HPMC) | 9.00 |
| Ethyl cellulose | 14.00 |
| Polyethylene oxide | 3.00 |
| Glycerol | 2.10 |
| FD&C Blue No. 1 (colorant) | 0.03 |
| Microcrystalline cellulose | 19.00 |
| Titanium dioxide | 1.00 |
| Levomenthol | 1.50 |
| Acesulfame K | 0.50 |
| Mint flavor | 0.50 |
| Nicotine bitartrate dihydrate | 3.07 |
| Isopropanol | 157.50 |
| Purified water | 28.35 |
| Total wet mass | 263.55 |
| Total dry mass | 77.70 |

Process: Nicotine bitartrate dihydrate, Acesulfame K, levomenthol and the FD&C Blue No. 1 are dissolved in the water-isopropanol mix. Glycerol, the liquid mint flavor, titanium dioxide and microcrystalline cellulose are then added. HPMC, ethyl cellulose, polyethylene oxide and PVP are added slowly under strong stirring. Slow stirring is kept until homogenization of the viscous mixture.

### Example 1e-2

Example 1e is repeated in an identical manner, except that the amount of nicotine bitartrate dihydrate used is 4.61 mg (=1.5 mg nicotine base equivalent) instead.

### Example 1e-3

Example 1e is repeated in an identical manner, except that the amount of nicotine bitartrate dihydrate used is 5.22 mg (=1.7 mg nicotine base equivalent) instead.

### Example 1e-4

Example 1e is repeated in an identical manner, except that the amount of nicotine bitartrate dihydrate used is 6.14 mg (=2.0 mg nicotine base equivalent) instead.

### Example 1f

### Preparation of a Casting Mixture Containing Nicotine Bitartrate Dihydrate [for Zones (a), (a′)]

| Ingredients | Amount (mg) |
|---|---|
| PVP | 24.000 |
| Eudragit ® NE 30 D (a 30% aqueous suspension) | 10.000 |
| Ethyl cellulose | 14.000 |
| Polyethylene glycol 400 | 0.800 |
| FD&C Blue No. 1 (colorant) | 0.050 |
| Microcrystalline cellulose | 16.000 |
| Titanium dioxide | 1.000 |
| Levomenthol | 1.500 |
| Acesulfame K | 0.500 |
| Mint flavor | 0.500 |
| Nicotine bitartrate dihydrate | 3.07 |
| Isopropanol | 157.500 |
| Total wet mass | 228.92 |
| Total dry mass | 64.42 |

Process: Nicotine bitartrate dihydrate, Acesulfame K, levomenthol and FD&C Blue No. 1 are dissolved in isopropanol. Polyethylene glycol 400, liquid mint flavor, Eudragit® NE 30 D, titanium dioxide and microcrystalline cellulose are then added. Ethyl celullose and PVP are added slowly under strong stirring. Slow stirring is kept until homogenization of the viscous mixture.

### Example 1f-2

Example 1f is repeated in an identical manner, except that the amount of nicotine bitartrate dihydrate used is 4.61 mg (=1.5 mg nicotine base equivalent) instead.

### Example 1f-3

Example 1f is repeated in an identical manner, except that the amount of nicotine bitartrate dihydrate used is 5.53 mg (=1.8 mg nicotine base equivalent) instead.

### Example 1f-4

Example 1f is repeated in an identical manner, except that the amount of nicotine bitartrate dihydrate used is 6.14 mg (=2.0 mg nicotine base equivalent) instead.

### Example 2

### Preparation of a Casting Mixture Containing Sodium Carbonate (with Croscarmellose Sodium) [for Zones (b), (b′)]

| Ingredients | Amount (mg) |
|---|---|
| Methocel E50 (HPMC) | 30.00 |
| Croscarmellose sodium | 6.00 |
| Glycerol | 3.00 |
| Xanthan gum | 1.00 |
| Sodium carbonate | 8.00 |
| Menthol | 1.50 |
| Acesulfame K | 0.50 |
| Mint flavor | 1.00 |
| Total dry mass | 51.00 |
| Purified water | 220.00 |
| Ethanol 96% | 140.00 |
| Total wet mass | 411.00 |

Process: Sodium carbonate, Acesulfame K and menthol are dissolved in water. Glycerol and ethanol are then added (sodium carbonate precipitation). HPMC, croscarmellose sodium and xanthan gum are added slowly under strong stirring. Slow stirring is kept until homogenization of the viscous mixture.

### Example 2d

### Preparation of a Casting Mixture Containing Sodium Carbonate [for Zones (b), (b′)]

| Ingredients | Amount (mg) |
|---|---|
| PVP | 24.00 |
| Metolose 60 SH 50 (HPMC) | 9.00 |
| Ethyl cellulose | 18.00 |
| Glycerol | 2.10 |
| Sodium carbonate | 9.00 |
| Microcrystalline cellulose | 15.00 |
| Levomenthol | 1.50 |
| Acesulfame K | 0.50 |
| Mint flavor | 0.50 |
| Isopropanol | 150.00 |
| Purified water | 27.00 |
| Total wet mass | 259.67 |
| Total dry mass | 82.67 |

Process: Acesulfame K and levomenthol are dissolved in the water-isopropanol mix. Glycerol, liquid mint flavor, titanium dioxide, sodium carbonate and microcrystalline cellulose are then added. HMPC, ethyl celullose and PVP are added slowly under strong stirring. Slow stirring is kept until homogenization of the viscous mixture.

### Example 2d-2

Example 2d is repeated in an identical manner, except that the amount of sodium carbonate used is 13.5 mg instead.

### Example 2d-3

Example 2d is repeated in an identical manner, except that the amount of sodium carbonate used is 15.3 mg instead.

### Example 2d-4

Example 2d is repeated in an identical manner, except that the amount of sodium carbonate used is 18.0 mg instead.

### Example 2e

### Preparation of a Casting Mixture Containing Sodium Carbonate [for Zones (b), (b′)]

| Ingredients | Amount (mg) |
|---|---|
| PVP | 24.00 |
| Metolose 60 SH 50 (HPMC) | 9.00 |
| Ethyl cellulose | 14.00 |
| Polyethylene oxide | 3.00 |
| Glycerol | 2.10 |
| Sodium carbonate | 9.00 |
| Microcrystalline cellulose | 10.00 |
| Titanium dioxide | 1.00 |
| Levomenthol | 1.50 |
| Acesulfame K | 0.50 |
| Mint flavor | 0.50 |
| Isopropanol | 157.50 |
| Purified water | 28.35 |
| Total wet mass | 260.45 |
| Total dry mass | 74.60 |

Process: Acesulfame K and levomenthol are dissolved in the water-isopropanol mix. Glycerol, the liquid mint flavor, titanium dioxide, sodium carbonate and microcrystalline cellulose are then added. HPMC, ethyl cellulose, polyethylene oxide and PVP are added slowly under strong stirring. Slow stirring is kept until homogenization of the viscous mixture.

### Example 2e-2

Example 2e is repeated in an identical manner, except that the amount of sodium carbonate used is 13.5 mg instead.

### Example 2e-3

Example 2e is repeated in an identical manner, except that the amount of sodium carbonate used is 16.2 mg instead.

### Example 2e-4

Example 2e is repeated in an identical manner, except that the amount of sodium carbonate used is 18.0 mg instead.

### Example 2f

### Preparation of a Casting Mixture Containing Sodium Carbonate [for Zones (b), (b′)]

| Ingredients | Amount (mg) |
|---|---|
| PVP | 24.00 |
| Eudragit ® NE 30 D (a 30% aqueous suspension) | 10.00 |
| Ethyl cellulose | 14.00 |
| Polyethylene glycol 400 | 0.80 |
| Sodium carbonate | 9.00 |
| Microcrystalline cellulose | 10.00 |
| Titanium dioxide | 1.00 |
| Levomenthol | 1.50 |
| Acesulfame K | 0.50 |
| Mint flavor | 0.50 |
| Isopropanol | 157.50 |
| Total wet mass | 228.80 |
| Total dry mass | 64.30 |

Process: Acesulfame K and levomenthol are dissolved in isopropanol. Polyethylene glycol 400, liquid mint flavor, Eudragit® NE 30 D, titanium dioxide, sodium carbonate and microcrystalline cellulose are then added. Ethyl cellulose and PVP are added slowly under strong stirring. Slow stirring is kept until homogenization of the viscous mixture.

### Example 2f-2

Example 2f is repeated in an identical manner, except that the amount of sodium carbonate used is 13.5 mg instead.

### Example 2f-3

Example 2f is repeated in an identical manner, except that the amount of sodium carbonate used is 15.3 mg instead.

### Example 2f-4

Example 2f is repeated in an identical manner, except that the amount of sodium carbonate used is 18.0 mg instead.

### Example 3

### Joint Coating of Casting Mixtures Both of Example 1c and Example 2 to Form Single, Integral Film Sheet that is Longitudinally Divided into Eight Stripes

A PET web liner is used as carrier for slot die coating to obtain a dry film sheet of 120 micrometer thickness with alternating four stripes composed of the casting mixture of example 1c and four stripes composed of the casting mixture of example 2. The slot die coating device is designed so that the width of each of the eight stripes is 15 mm. The joint coating is done with an adapted casting mixture pump speed for each product in order to reach 15.4 mg/cm² of dry coat weight for product 1c and 17 mg/cm² of dry coat weight for product of example 2. The web speed is set at 0.3 m/minute. Drying of the film sheet is carried out with an infra-red preheating and at 60° C. in a drying oven. The dried film sheet is coiled up on a bulk master roll.

### Example 4

### Cutting of Film Sheet of Example 3 so as to Obtain Bi- or Multi-Zone Single-Layer Films (30 mm×20 mm) Comprising 3.07 mg of Nicotine Bitartrate Dihydrate (=1 mg Nicotine Base Equivalent) and 9 mg of Sodium Carbonate

Films of 30 mm width (=twice the width of one stripe of the film sheet) and 20 mm length are cut from the film sheets of Example 3 by die cutting. The cutting need not necessarily be perfectly aligned to the stripes (cp. FIG. 3, films “2”, “3” and “4”). All films have the same amount of nicotine active and alkaline substance in one unit, namely 3.07 mg of nicotine bitartrate dihydrate and 9 mg of sodium carbonate.

### Example 5

Joint coating of casting mixtures both of Example 1d and Example 2d to form single, integral film sheet that is longitudinally divided into eight stripes is done essentially as described in Example 3: A PET web liner is used as carrier for slot die coating to obtain a dry film sheet of about 60 micrometer thickness with alternating four stripes composed of the casting mixture of example 1c and four stripes composed of the casting mixture of example 2. The slot die coating device is designed so that the width of each of the eight stripes is 22 mm. The joint coating is done with an adapted casting mixture pump speed for each product in order to reach 25.1 mg/cm² of dry coat weight for both 1d and 2d products. Drying of the film sheet is carried out with a drying oven. The dried film sheet is coiled up on a bulk master roll.

### Example 5a

Example 5 is repeated, except that the slot die coating device is designed so that there are obtained 16 stripes with a width of 11 mm each.

### Example 6

### Cutting of Film Sheet of Example 5 so as to Obtain Bi- or Multi-Zone Single-Layer Films (30 mm×22 mm) Comprising 3.07 mg of Nicotine Bitartrate Dihydrate (=1 mg Nicotine Base Equivalent) and 9 mg of Sodium Carbonate

Films of 22 mm width (=the width of one stripe of the film sheet) and 30 mm length are cut from the film sheets of Example 5 by die cutting. In this case, the cutting must be done very accurately from the middle of one zone (e.g. “b” in FIG. 3) to the middle of the other zone (e.g. “a” in FIG. 3) to ensure that the exact amounts of both nicotine and alkaline substance (3.07 mg of nicotine bitartrate dihydrate and 9 mg of sodium carbonate) are present in each film.

### Example 6a

Example 6 is repeated, except that films of 22 mm width (=twice the width of one stripe of the film sheet) and 30 mm length are cut from the film sheets of Example 5a by die cutting. Here the cutting need not necessarily be perfectly aligned to the stripes (cp. FIG. 3, films “2”, “3” and “4”). All films have the same amount of nicotine active and alkaline substance in one unit, namely 3.07 mg of nicotine bitartrate dihydrate and 9 mg of sodium carbonate.

### Example 7

Joint coating of casting mixtures both of Example 1e and Example 2e to form single, integral film sheet that is longitudinally divided into eight stripes is done essentially as described in Example 5. Likewise, the cutting of the film sheet so as to obtain bi- or multi-zone single-layer films (30 mm×22 mm) comprising 3.07 mg of nicotine bitartrate dihydrate (=1 mg nicotine base equivalent) and 9 mg of sodium carbonate is done essentially as described in Example 6.

### Example 8

Joint coating of casting mixtures both of Example 1f and Example 2f to form single, integral film sheet that is longitudinally divided into 16 stripes is done essentially as described in Example 5a. Likewise, the cutting of the film sheet so as to obtain bi- or multi-zone single-layer films (30 mm×22 mm) comprising 3.07 mg of nicotine bitartrate dihydrate (=1 mg nicotine base equivalent) and 9 mg of sodium carbonate is done essentially as described in Example 6a.

### Example 9

Joint coating of casting mixtures both of Example 1f-2 and Example 2f-2 to form single, integral film sheet that is longitudinally divided into 16 stripes is done essentially as described in Example 5a. Likewise, the cutting of the film sheet so as to obtain bi- or multi-zone single-layer films (30 mm×22 mm) comprising 4.61 mg of nicotine bitartrate dihydrate (=1.5 mg nicotine base equivalent) and 13.5 mg of sodium carbonate is done essentially as described in Example 6a.

### Example 10

Joint coating of casting mixtures both of Example 1f-2 and Example 2f-4 to form single, integral film sheet that is longitudinally divided into 16 stripes is done essentially as described in Example 5a. Likewise, the cutting of the film sheet so as to obtain bi- or multi-zone single-layer films (30 mm×22 mm) comprising 4.61 mg of nicotine bitartrate dihydrate (=1.5 mg nicotine base equivalent) and 18 mg of sodium carbonate is done essentially as described in Example 6a.

1. A single-layer, oral disintegrating film having at least two distinct zones,
wherein at least one zone (a) consists of a first composition that comprises a nicotine active, and
wherein at least one zone (b) consists of a second composition that comprises an alkaline substance,
and which oral disintegrating film optionally includes one or more further distinct zones selected from the group of zones (a′) and (b′), which zones (a′) and (b′) are characterized by having essentially the same compositions as the corresponding zones (a) and (b), respectively.

2. A film according to claim 1, which adheres to the buccal mucosa.

3. A film according to claim 1, which disintegrates completely within 1 to 10 min after being put into the mouth of a patient.

4. A film according to claim 1, which provides for a concerted release of both nicotine active and alkaline substance, thereby ensuring efficient and fast buccal absorption of nicotine free base.

5. A film according to claim 1, wherein the alkaline substance is selected from the group consisting of sodium carbonate, potassium carbonate, sodium bicarbonate, potassium bicarbonate, sodium hydroxide, potassium hydroxide and any mixtures thereof.

6. A film according to claim 1, wherein the nicotine active is selected from the group consisting of pharmaceutically acceptable nicotine salts, pharmaceutically acceptable nicotine complexes and pharmaceutically acceptable nicotine resinates.

7. A film according to claim 6, wherein the nicotine active is a pharmaceutically acceptable salt of nicotine.

8. A film according to claim 7, wherein the first composition forming zones (a) and (a′) further comprises at least one water-soluble film-forming polymer,
and wherein the second composition forming zones (b) and (b′) further comprises at least one water-soluble film-forming polymer.

9. A film according to claim 8, wherein the at least one water-soluble film-forming polymer in the first composition forming zones (a) and (a′) is a mixture of at least one water soluble film forming polymer selected from the group consisting of cellulose ether derivatives and at least one water soluble film forming polymer selected from the group consisting of synthetically or naturally occurring gums and polyalkylene oxides; and
wherein the at least one water-soluble film-forming polymer in the second composition forming zones (b) and (b′) is a mixture of at least one water soluble film forming polymer selected from the group consisting of cellulose ether derivatives and at least one water soluble film forming polymer selected from the group consisting of synthetically or naturally occurring gums.

10. A film according to claim 9, wherein the second composition further comprises a polymer selected from the group consisting of croscarmellose sodium, corn starch, and any mixtures thereof.

11. A film according to claim 10, wherein the first composition further comprises a polymer selected from the group consisting of croscarmellose sodium, corn starch, and any mixtures thereof.

12. A film according to claim 1, wherein coloring agents are added to either the first or the second composition or both, resulting in a film wherein the color of the zones (b) and optionally (b′) is different from the color of the zones (a) and optionally (a′), wherein the color of the zones (a) and optionally (a′) is the same, and wherein the color of the zones (b) and optionally (b′) is the same.

13. A single, integral film sheet which is longitudinally divided at least once into at least one stripe composed of a first composition that comprises a nicotine active and another at least one stripe composed of a second composition comprising an alkaline substance; wherein the different compositions composing each stripe have merged together.

14. A double-or multilayer film sheet comprising a single, integral film sheet according to claim 13 and at least a second layer intimately attached to said film sheet.

15. A method of preparing an oral disintegrating film according to claim 1, the method comprising the steps of:
mixing all components intended to form zones (a) and optionally (a′) to form a first homogenous mixture;
mixing all components intended to form zones (b) and optionally (b′) to form a second homogenous mixture;
feeding said first and second homogenous mixtures separately via at least two different inlets into a coating device capable of spreading each composition into a uniform thin layer while allowing for the separate zones to interface, creating one integral coating with at least two unique stripes;
whereby a film is obtained that is longitudinally divided at least once into at least one stripe composed of said first homogenous mixture and another at least one stripe composed of said second homogenous mixture; the different coating mixes composing each zone do merge together during the casting into a neat interface and forming a single, integral film sheet;
drying the cast film; and cutting the produced film into oral disintegrating films such that each oral disintegrating film has at least two distinct zones, at least one of which is composed of said first homogenous mixture and at least one other of which is composed of said second homogenous mixture.
